(19) United States
(12) Patent Application Publication
(10) Pub. No.: US 2018/0177860 A1
(43) Pub. Date: Jun. 28, 2018

(54) VACCINE CONTAINING VIRUS INACTIVATED BY GREEN TEA EXTRACT, AND PREPARATION METHOD THEREFOR

(71) Applicant: INDUSTRY-ACADEMIC COOPERATION FOUNDATION, YONSEI UNIVERSITY, Seoul (KR)

(72) Inventors: Baik Lin SEONG, Chungcheongnam-do (KR); Yun Ha LEE, Chungcheongnam-do (KR)

(21) Appl. No.: 15/735,449

(22) PCT Filed: Jun. 7, 2016
(86) PCT No.: PCT/KR2016/005989

§ 371 Date: Dec. 11, 2017

(30) Foreign Application Priority Data
Jun. 11, 2015 (KR) 10-2015-0082653

(51) Int. Cl. A61K 39/145; A61K 39/215; A61K 39/12

(52) U.S. Cl. CPC A61K 39/145; A61K 2039/5252; A61K 39/12; A61K 39/215

(57) ABSTRACT

The present invention relates to a vaccine composition containing a virus inactivated by a green tea extract, and a preparation method therefor. According to the present invention, when a virus is treated with a green tea extract, there are simultaneous effects of virus inactivation and excellent immunogenicity maintenance, and thus an inactivated vaccine can be prepared by mixing the green tea extract of the present invention and a virus with a proliferative capacity, and infectious diseases caused by the corresponding virus can be effectively prevented since an immune reaction to the corresponding virus is induced when a vaccine composition prepared by the preparation method of the present invention is administered to a subject. In addition, there are advantages of enabling the preparation of a safe virus vaccine since the green tea extract of the present invention is nontoxic, and a preparation process is economical since, unlike a chemical material-based preparation process, a dialysis process is unnecessary.

### TECHNICAL FIELD

The present invention was made with the support of the Ministry of Health and Welfare, Republic of Korea, under Project No. HI13C0826, which was conducted in the program titled “Vaccine Translational Research Center” in the project named “Development of infectious disease crisis response technology”, by the Industry-Academic Cooperation Foundation, YONSEI University, under the management of the Korea Health Industry Development Institute, from 24 Jun. 2013 to 23 Jun. 2018.

The present invention was made with the support of the Ministry of Health and Welfare, Republic of Korea, under Project No. HI15C2934, which was conducted in the program titled “Green tea catechin-based improved inactivated virus vaccine development” in the project named “Development of infectious disease crisis response technology”, by the Industry-Academic Cooperation Foundation, YONSEI University, under the management of the Korea Health Industry Development Institute, from 3 Dec. 2015 to 30 Nov. 2016.

This application claims priority to and the benefit of Korean Patent Application No. 10-2015-0082653 filed in the Korean Intellectual Property Office on 11 Jun. 2015, the entire contents of which are incorporated herein by reference.

The present invention relates to a vaccine containing a virus inactivated by a green tea extract, and a preparation method therefor.

### BACKGROUND

An attenuated vaccine (live vaccine) that contains replicative viruses with low pathogenicity, has an advantage of inducing a humoral immune response as well as a cellular immune response in a subject to which the vaccine has been administered. However, the attenuated vaccine contains replicative viruses, and thus, the attenuated vaccine is likely to recover pathogenicity thereof due to back-mutation with circulation in the population. It is difficult to maintain the infectivity of the attenuated vaccine during storage and transport. Whereas, an inactivated vaccine (killed vaccine) contains a dead virus, and thus, the pathogenicity thereof cannot be recovered and the contamination of other living microorganisms does not occur in the inactivated vaccine, and therefore, the inactivated vaccine is more safe than the attenuated vaccine (Bardiya, N. et al., 2005. Influenza vaccines: recent advances in production technologies. Applied microbiology and biotechnology 67, 299-305). In addition, the inactivated vaccine is produced by treating the replicative viruses with heat, UV, formalin (formaldehyde), binary ethylenimine (BEI), and β-propiolactone (Goldstein M A et al., Effect of formalin, beta-propiolactone, merthiolate, and ultraviolet light upon influenza virus infectivity chicken cell agglutination, hemagglutination, and antigenicity. Appl Microbiol. 1970 February; 19(2):290-). The development period is relatively short and efficient for mass production, and very economical, and therefore, the method has been routinely used for vaccine development.

Formaldehyde is one of the most generally used inactivating agents for the production of inactivated vaccines. Formaldehyde has very strong toxicity, so 30 ml of 37% formaldehyde can lead to death in adults. Formaldehyde is absorbed by inspiration or through the skin or eyes, and may cause symptoms, such as headache and dyspnea, and may cause damage to the respiratory tract. Therefore, the inoculation of vaccines inactivated with formaldehyde into our body may cause hypersensitivity and side effects due to residual formaldehyde.

In addition, formaldehyde can easily inactivate viruses, and because the viral protein is fixed, the immune response can be easily induced in the subject to which the vaccine is administered. Poliovirus, however, is known to undergo partial modifications in the antigenic structure during formaldehyde treatment (Ferguson, M. et al., 1993. Antigenic structure of poliovirus in inactivated vaccines. The Journal of general virology 74(Pt 4), 685-690002E). Therefore, an effective immune response may not occur. In some cases, among individuals infected with respiratory syncytial virus (RSV) or measles virus, very severe symptoms were shown in individuals who have been vaccinated with formaldehyde-inactivated vaccines as compared with unvaccinated individuals. The reason is that vaccine inoculation has increased the susceptibility to viral infection, and this fact is known to be associated with residual formaldehyde in the body after vaccination. Moghaddam, A. and et al., showed the results that the increased TH2 response and strongly induced IL-4 and IL-5 were observed in mice inoculated with formalin-inactivated RSV (2006. A potential molecular mechanism for hypersensitivity caused by formalin-inactivated vaccines. Nature medicine 12, 905-907, Openshaw, P. J. et al., 2001. Immunopathogenesis of vaccine-enhanced RSV disease. Vaccine 20 Suppl 1, S27-31). In addition, formaldehyde needs to be removed through dialysis after the inactivation process in the production of formaldehyde-inactivated vaccines (Furuya, Y. et al., 2010. Effect of inactivation method on the cross-protective immunity induced by whole “killed” influenza A viruses and commercial vaccine preparations. Journal of General Virology 91, 1450-1460. Andrew, S. M. et al., 2001. Dialysis and concentration of protein solutions. Current protocols in immunology/edited by John E. Coligan. [et al.], Appendix 3H), and thus, there are a lot of additional production costs for dialysis, besides virus purification and inactivation. Therefore, there is a need to develop new inactivating agents that can replace formaldehyde.

The present inventors selected a green tea extract in order to inactivate viruses. Green tea is produced from a plant called Camellia sinensis, and is often used as beverages, or applied as a diet food or a cosmetic product (Cabrera, C. et al., Beneficial effects of green tea review. Journal of the American College of Nutrition 25, 79-99). An extract of green tea is composed of several kinds of catechins, specifically, (−)-epigallocatechin (EGC), (−)-epicatechin gallate (ECG), (−)-epigallocatechin gallate (EGCG), and (−)-epicatechin (EC). Of these, epicatechin gallate (EGCG), which is a main catechin, is known to inhibit intracellular invasion of several viruses and prevent the cell adhesion thereof (Colpitts, C. C. et al., 2014. A small molecule inhibits virion attachment to heparan sulfate- or sialic acid-containing glycans. Journal of virology 88, 7806-7817). Especially, it has been reported that epicatechin gallate (EGCG) inhibits neuraminidase activity in influenza viruses and shows anti-viral effects in cells during the infection, replication, and then release steps (Song, J. M. et al., 2005. Antiviral effect of catechins in green tea on influenza virus. Antiviral research 68, 66-74). However, there are no cases in which a green tea extract is used for virus inactivation to produce vaccines.

Throughout the entire specification, many papers and patent documents are referenced, and their citations are represented. The disclosure of cited papers and patent documents is entirely incorporated by reference into the present specification, and the level of the technical field within which the present invention falls and details of the present invention are explained more clearly.

### DETAILED DESCRIPTION

### Technical Problem

The present inventors researched and endeavored to solve problems associated with toxic chemical substance (e.g., formaldehyde)-based inactivation method that has been already used in the preparation of inactivated virus vaccines. As a result, the present inventors verified that the treatment a virus with a green tea extract achieves complete and irreversible inactivation of the virus and maintenance of immunogenicity of the virus, is nontoxic, and also has excellent defensive ability, thus, completed the present invention.

Therefore, an aspect of the present invention is to provide a vaccine composition containing a virus inactivated by a green tea extract.

Another aspect of the present invention is to provide a method for preparing an inactivated virus vaccine, the method including: (a) adding a green tea extract to a replicative virus, followed by mixing; and (b) incubating a mixture of the virus and the green tea extract.

Still another aspect of the present invention is to provide a method for preventing a viral infectious disease, the method including administering the vaccine composition to a subject.

Other purposes and advantages of the present disclosure will become more obvious with the following detailed description of the invention, claims, and drawings.

### Technical Solution

The present inventors researched and endeavored to solve problems in a chemical substance (e.g., formaldehyde)-based inactivation method that has been already used in the preparation of inactivated virus vaccines. As a result, the present inventors verified that the virus treated with a green tea extract was completely and irreversibly inactivated, maintained immunogenicity of the virus, was not toxic and had excellent defense against viruses.

Therefore, the present invention is directed to i) a vaccine composition containing a virus inactivated by a green tea extract, ii) a method for preparing an inactivated virus vaccine, the method including: adding a green tea extract to a replicative virus, followed by mixing; and incubating a mixture of the virus and the green tea extract, and iii) a method for preventing a viral infectious disease, the method including administering the vaccine composition to a subject.

In accordance with an aspect of the present invention, there is provided a vaccine composition containing a virus inactivated by a green tea extract.

As used herein, the term “green tea extract” may be obtained by using, as an extraction solvent, various extraction solvents, for example, (a) water, (b) a C1-C4 anhydrous or hydrous lower alcohol (methanol, ethanol, propanol, butanol, etc.), (c) a mixed solvent of the lower alcohol with water, (d) acetone, (e) ethyl acetate, (f) chloroform, (g) 1,3-butylene glycol, and (h) butyl acetate. According to an embodiment of the present invention, the green tea extract of the present invention is obtained by using water as an extraction solvent. According to another embodiment of the present invention, the green tea extract of the present invention contains several kinds of catechins, specifically contains (−)-epigallocatechin (EGC), (−)-epicatechin gallate (ECG), (−)-epigallocatechin gallate (EGCG), and (−)-epicatechin (EC), and most specifically contains (−)-epigallocatechin gallate (EGCG). According to still another embodiment of the present invention, the green tea extract of the present invention may be obtained by using ethanol as an extraction solvent, and according to a particular embodiment of the present invention, the green tea extract may be obtained by using 70% ethanol as an extraction solvent. Meanwhile, it would be obvious that an extract showing substantially the same effect as the extract of the present invention may be obtained by using, besides the extraction solvents, even other different extraction solvents.

As used herein, the term “extract” has a meaning that is commonly used as a crude extract in the art as described above, and in a broad sense, the term also includes a fraction obtained by additionally fractionating the extract. In other words, the green tea extract of the present invention includes not only ones obtained by using the foregoing extraction solvents but also ones obtained by additionally applying a purification procedure to the same. For example, the extract of the present invention also includes fractions obtained by passing the extract through an ultrafiltration membrane with a cut-off value of a predetermined molecular weight, and fractions obtained through various purification methods that are further carried out, such as separation by various chromatographies (manufactured for separation depending on size, charge, hydrophobicity, or hydrophilicity). The extract of the present invention also includes ones that are prepared into a powder state by additional procedures, such as distillation under reduced pressure and freeze-drying or spray drying.

As used herein, the “vaccine” is used in a broadest sense to refer to a composition that positively affects an immune response of a subject. The vaccine composition provides the subject with a cellular immune response such as cytotoxic T lymphocyte (CTL), or a humoral immune response such as an enhanced systemic or local immune response induced by an antibody.

According to an embodiment of the present invention, the virus of the present invention is an enveloped virus or a non-enveloped virus. The enveloped virus of the present invention includes, but is not limited to, Poxviridae (e.g., vaccinia and smallpox), Iridoviridae, Herpesviridae (e.g., herpes simplex, varicella virus, cytomegalovirus, and Epstein-Barr virus), Flaviviridae (e.g., yellow fever virus, Tick-borne encephalitis virus, and hepatitis C virus), Togaviridae (e.g., Rubella virus and Sindbis virus), Coronaviridae [e.g., human coronavirus (severe acute respiratory syndrome (SARS) virus), avian infectious bronchitis virus (IBV)], Paramyxoviridae (e.g., parainfluenza virus, mumps virus, measles virus, and respiratory syncytial virus), Rabdoviridae (e.g., vesicular stomatitis virus and rabies viruses), Filoviridae (e.g., Marburg virus and Ebola virus), Orthomyxoviridae (e.g., influenza A and B viruses), Bunyaviridae (e.g., Bwamba virus, California encephalitis virus, sandfly fever virus, and valley fever virus), Arenaviridae (e.g., LCM virus, Lassa virus, and Junin virus), Hepadnaviridae (e.g. hepatitis B virus), and Retroviridae (e.g., HTLV and HIV). The non-enveloped virus of the present invention includes, but is not limited to, norovirus, rotavirus, adenovirus, poliovirus, and reovirus. According to another embodiment of the present invention, the virus of the present invention is an enveloped virus. According to a specific embodiment of the present invention, the virus of the present invention is an influenza virus. The influenza virus of the present invention includes influenza viruses capable of infecting mammals or birds, and examples thereof include, but are not limited to, birds, people, dogs, horses, pigs, cats, and the like. The influenza virus of the present invention includes the influenza virus itself and various influenza virus-derived antigens that are conventionally known. The antigen refers to an antigen component capable of causing an immune function among viral components. According to a specific embodiment of the present invention, the antigen includes nucleoprotein (NP), hemagglutinin (HA), neuraminidase (NA) or fragments thereof. According to another embodiment of the present invention, the influenza virus of the present invention is influenza A virus, influenza B virus, or influenza C virus. According to a certain embodiment of the present invention, the influenza virus of the present invention is influenza A virus, an example of which is A/H1N1, A/H3N2, A/H5N2, or A/H9N2 virus. According to a specific embodiment of the present invention, the A/H1N1 virus of the present invention is A/Puerto Rico/8/34 (H1N1) virus, A/Chile/1/83 (H1N1) virus, A/NWS/33 virus, or A/Korea/01/2009 (H1N1) virus. According to another specific embodiment of the present invention, the A/H3N2 virus of the present invention is A/Sydney/5/97 (H3N2), and A/H5N2 is A/Aquatic bird/Korea/w81/05 (H5N2), and A/H9N2 is A/chicken/Korea/310/01 (H9N2). The influenza virus may cause flu, cold, a sore throat, bronchitis, or pneumonia in humans, and especially, may cause bird flu, swine flu, or goat flu.

According to a certain embodiment of the present invention, the virus of the present invention is a coronavirus. The coronavirus of the present invention includes coronavirus, which infects animals, such as humans, mammals, and birds, to cause diseases in respiratory or gastrointestinal tracts, and may infect hosts to cause clinical symptoms, such as weight loss, runny nose, fever, cough, headache, and diarrhea. The coronavirus includes, but is not limited to, severe acute respiratory syndrome virus (SARS-CoV), middle east respiratory syndrome virus (MERS-CoV), infectious bronchitis virus (IBV), swine transmissible gastroenteritis virus (TGE), swine flu epidemic diarrhea virus (PED), bovine coronavirus (BCoV), feline/canine coronavirus (FCoV/CCoV), mouse hepatitis virus (MHV), and the like. According to a specific embodiment of the present invention, the coronavirus of the present invention is infectious bronchitis virus (IBV) strain M41, which belongs to the same genus as and has genetic similarity to severe acute respiratory syndrome virus (SARS-CoV), occurring in Asia in 2003 and spread worldwide to cause nearly 800 deaths, and middle east respiratory syndrome virus (MERS-CoV), bring about infected people in the Middle East, mainly in Saudi Arabia, the United Arab Emirates, Jordan, and Qatar, and more than 100 infected people across Korea from May 2015 (Travis R. Ruch et. al., 2012. The Coronavirus E Protein: Assembly and Beyond. Viruses 4(3), 363-382/Xing-Yi Ge et al., 2013. Isolation and characterization of a bat SARS-like coronavirus that uses the ACE2 receptor. Nature 503, 535-538).

According to a certain embodiment of the present invention, the virus of the present invention is human papillomavirus. The human papillomavirus of the present invention is a kind of virus that causes warts in humans, and there are more than 100 species of human papillomavirus. The human papillomavirus infects the skin surface, causing warts on hands, feet, and genital mucosa, and may cause cervical cancer in women. According to a particular embodiment of the present invention, the human papillomavirus may be specifically HPV type 16, 18, 31, 33, 35, 39, 45, 51, 52, 56, 58, 59, or 66, and more specifically, HPV type 16.

According to a certain embodiment of the present invention, the virus of the present invention is norovirus. The norovirus of the present invention causes severe nausea, vomiting, diarrhea, abdominal pain, chills, fever of about 38° C., and the like in humans, and includes Norwalk virus.

According to one embodiment of the present invention, the green tea extract of the present invention binds to nucleoprotein or hemagglutinin of the influenza virus of the present invention. According to a certain embodiment of the present invention, the green tea extract of the present invention binds to a globular domain or a stalk region of hemagglutinin of the influenza virus of the present invention. According to another embodiment of the present invention, the green tea extract of the present invention binds to a globular domain or a stalk region of hemagglutinin of the virus of the present invention. As shown in the following examples, it can be seen that the treatment with a green tea extract increases the sizes of all influenza virus proteins (e.g., nucleoprotein, hemagglutinin full protein, and globular domain and stalk region of hemagglutinin) (FIGS. 1a-1d).

According to one embodiment of the present invention, the green tea extract of the present invention binds to coronavirus, human papillomavirus, and norovirus of the present invention. As shown in the following examples, it can be seen that the treatment with a green tea extract increases the sizes of all proteins of infectious bronchitis virus, human papillomavirus, and norovirus (FIGS. 1e, 13, and 14).

The vaccine composition of the present invention contains a pharmaceutically effective amount of virus inactivated by the green tea extract of the present invention, and the green tea extract binds to viral proteins. The vaccine composition of the present invention may further contain a pharmaceutically acceptable carrier. As used herein, the term “pharmaceutically effective amount” refers to an amount sufficient to achieve preventive, alleviative, or therapeutic efficacy against a disease or pathological syndrome caused by virus infection. The pharmaceutically acceptable carriers that may be contained in the composition of the present invention are generally used in formulation. Examples of the pharmaceutically acceptable carrier include, but are not limited to, lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and mineral oil. The composition of the present invention may further contain, in addition to the above components, a lubricant, a wetting agent, a sweetening agent, a flavoring agent, an emulsifier, a suspending agent, a preservative, and the like. Suitable pharmaceutically acceptable carriers and agents are described in detail in Remington's Pharmaceutical Sciences (19th ed., 1995). The vaccine composition of the present invention may contain other components, such as a stabilizer, an excipient, other pharmaceutically acceptable compounds, or any other antigen or a portion thereof. The vaccine may be present in the form of a freeze-dried preparation or a suspension, all of which are common in the field of vaccine production.

The dosage form of the vaccine composition of the present invention may be in the form of an enteric-coated use unit, or inoculation for intraperitoneal, intramuscular, or subcutaneous administration, aerosol spray, oral, or intranasal use. The vaccine composition may be administered as drinking water or an edible pellet. The vaccine composition of the present invention may also be transferred as a single vaccine in which immunomodulatory molecules, such as heterologous antigens and cytokines, are expressed in the same recombinant, and may be administered as “a cocktail” which contains two or more viral vectors carrying different foreign genes or an adjuvant. As used herein, the term “adjuvant” generally refers to any material (e.g., alum, Freund's complete adjuvant, Freund's incomplete adjuvant, LPS, poly IC, poly AU, etc.) that increases body fluids or cellular immune responses to antigens.

In accordance with another aspect of the present invention, there is provided a method for preparing an inactivated virus vaccine, the method including: (a) adding a green tea extract to a replicative virus, followed by mixing; and (b) incubating a mixture of the virus and the green tea extract.

According to one embodiment of the present invention, the inactivated virus vaccine of the present invention contains a virus inactivated by a green tea extract, and the green tea extract binds to viral proteins.

According to one embodiment of the present invention, the inactivated virus of the present invention is an influenza virus, an example of which is influenza A virus, influenza B virus, or influenza C virus. According to a certain embodiment of the present invention, the influenza virus of the present invention is influenza A virus, an example of which is A/H1N1, A/H3N2, A/H5N2, or A/H9N2 virus. According to a specific embodiment of the present invention, the A/H1N1 virus of the present invention is A/Puerto Rico/8/34 (H1N1) virus, A/Chile/1/83 (H1N1) virus, A/NWS/33 virus, or A/Korea/01/2009(H1N1) virus. According to another specific embodiment of the present invention, the A/H3N2 virus of the present invention is A/Sydney/5/97 (H3N2), and A/H5N2 is A/Aquatic bird/Korea/w81/05 (H5N2) and A/H9N2 is A/chicken/Korea/310/01 (H5N2).

According to an embodiment of the present invention, the influenza virus and the green tea extract of the present invention are mixed at a ratio of 5×10¹⁰ to 5×10³ PFU:0.1-100 mg. As shown in the following examples, when 5×10⁸ to 5×10⁷ PFU/ml influenza virus was treated with 0.01-1 mg/ml green tea extract, virus replication activity and hemagglutination activity were reduced; when 1×10⁸ to 5×10⁷ PFU/ml influenza virus was mixed with 1 mg/ml green tea extract in equal amounts, virus replication activity was completely inhibited; and when 5×10⁷ PFU/ml influenza virus was mixed with 1 mg/ml green tea extract in equal amounts, both virus replication activity and hemagglutination activity were completely inhibited (FIG. 2b).

According to another embodiment of the present invention, the inactivated virus of the present invention is coronavirus. According to a certain embodiment of the present invention, the coronavirus includes coronaviruses capable of infecting humans and birds, and includes infectious bronchitis virus, SARS virus, and MERS virus. According to a particular embodiment of the invention, the coronavirus is infectious bronchitis virus strain M14.

According to an embodiment of the present invention, the coronavirus and the green tea extract of the present invention are mixed at a ratio of 10¹⁰-10³ EID₅₀:0.1-100 mg. As shown in the following example, it was confirmed that when 10⁶⁵ to 5×10^{5.5} EID₅₀/ml influenza virus was treated with 0.1-1 mg/ml green tea extract, virus activity was terminated in the allantoic fluid collected after the inoculation into chicken embryos (FIG. 10).

According to another embodiment of the present invention, the inactivated virus of the present invention is human papillomavirus. The human papillomavirus of the present invention is a kind of virus that causes warts in humans, and there are more than 100 species of human papillomavirus. The human papillomavirus infects the skin surface to cause warts on hands, feet, and genital mucosa, and may cause cervical cancer in women. According to a particular embodiment of the present invention, the human papillomavirus may be specifically HPV type 16, 18, 31, 33, 35, 39, 45, 51, 52, 56, 58, 59, or 66, and more specifically, may be HPV type 16.

According to another embodiment of the present invention, the inactivated virus of the present invention is norovirus. The norovirus causes severe nausea, vomiting, diarrhea, abdominal pain, chills, fever of about 38° C., and the like, in humans, and includes Norwalk virus.

As shown in an example of the present invention, it can be seen that the human papillomavirus and norovirus of the present invention were inactivated by allowing viral proteins to bind to the green tea extract (FIGS. 13 and 14).

According to an embodiment of the present invention, in the method of the present invention, the incubation is carried out at a temperature of 15° C. or higher after the step for mixing the virus and the green tea extract. According to a certain embodiment of the present invention, in the method of the present invention, the incubation is carried out at 15-50° C., 20-50° C., 25-50° C., 30-50° C., 33-50° C., 35-50° C., 15-45° C., 20-45° C., 25-45° C., 30-45° C., 33-45° C., 35-45° C., 15-40° C., 20-40° C., 25-40° C., 30-40° C., 33-40° C., 35-40° C., 15-38° C., 20-38° C., 25-38° C., 30-38° C., 33-38° C., 35-38° C., or 35° C. after the step for mixing the virus and the green tea extract, but is not limited thereto. The virus activity was lowered even when the incubation temperature was as low as 20° C. or lower, compared with a group treated without a green tea extract, and thus, it would be obvious that when the incubation was carried out at least at a temperature of 15-20° C. corresponding to a room temperature range, the virus replication activity was inhibited as in the example of the present invention, and thus the purpose of virus inactivation could be activated; and even when the incubation was carried out at a temperature of the above temperature range, the purpose of virus inactivation could be achieved. As shown in the following example, as a result of the treatment of an influenza virus with a green tea extract, followed by incubation at 20-35° C., virus replication activity and hemagglutination activity were reduced, and at 35° C., both influenza virus replication activity and hemagglutination activity were completely inhibited, and coronavirus activity was also terminated (FIGS. 2a and 10).

According to another embodiment of the present invention, the incubation of the present invention is carried out for 1 hour or longer. According to a certain embodiment of the present invention, the incubation of the present invention is carried out for 1-96 hours, 1-72 hours, 1-48 hours, 1-36 hours, 1-30 hours, 1-24 hours, 3-96 hours, 3-72 hours, 3-48 hours, 3-36 hours, 3-30 hours, 3-24 hours, 6-96 hours, 6-72 hours, 6-48 hours, 6-36 hours, 6-30 hours, or 6-24 hours. It would be obvious that when the incubation was carried out for at least 1 hour, virus replication was inhibited as in the example of the present invention, and thus the purpose of virus inactivation can be achieved; and even when the incubation was carried out for longer than 1 hour, the purpose of virus inactivation could be achieved. As shown in the following example, as a result of the treatment of a virus with a green tea extract, followed by incubation at 35° C., virus replication activity and hemagglutination activity began to decrease together with the initiation of incubation, and for the treatment with 1 mg/ml green tea extract, both virus replication activity and hemagglutination activity were completely inhibited by incubation within only 6 hours (FIG. 2c).

According to one embodiment of the present invention, the present invention may further include, after the incubation in step (b), (c) adding an excipient. As used herein, the term “excipient” has a meaning encompassing, in addition to the pharmaceutically acceptable carrier, a lubricant, a wetting agent, a sweetening agent, a flavoring agent, an emulsifier, a suspending agent, a preservative, and an adjuvant, and includes all excipients that are ordinarily used in the field associated with vaccine preparation.

According to one embodiment of the present invention, the present invention may further include, after the addition of the excipient in step (c), (d) performing filtration, sterilization, and dilution.

The filtration, sterilization, and dilution steps are a filtration step for removing foreign materials contained in the composition containing a virus inactivated by the green tea extract of the present invention, a sterilization step for sterilizing microbes (including viruses, germs, and molds) that may be incorporated in a vaccine container, besides the inactivated virus and the excipient, and a dilution step for diluting the composition containing the inactivated virus according to a pharmaceutically effective concentration, and all the filtration, sterilization, and dilution methods that are ordinarily used in the field associated with vaccine preparation of the present invention can be used without limitation.

In accordance with still another aspect of the present invention, there is provided a method for preventing a viral infectious disease, the method including administering the vaccine composition to a subject.

According to one embodiment of the present invention, the viral infectious disease is caused by an infection with an influenza virus, coronavirus, human papillomavirus, or norovirus.

The method for preventing a viral infectious disease of the present invention is associated with a method for using the foregoing vaccine composition, and thus, the description of overlapping contents therebetween will be omitted to avoid excessive complication of the specification.

### Advantageous Effects

The features and advantages of the present invention are summarized as follows:

(a) The present invention is directed to i) a vaccine composition containing a virus inactivated by a green tea extract, ii) a method for preparing an inactivated virus vaccine, the method including: adding a green tea extract to a replicative virus, followed by mixing; and incubating a mixture of the virus and the green tea extract, and iii) a method for preventing a viral infectious disease, the method including administering the vaccine composition to a subject.

(b) According to the present invention, the treatment of a virus with a green tea extract produces effects of achieving complete inactivation of the virus and excellent maintenance of immunogenicity of the virus. Therefore, an inactivated vaccine can be prepared by mixing the green tea extract according to the present invention and a replicative virus, and when the vaccine composition prepared by the method of the present invention is administered to a subject, an immune response against a corresponding virus is induced, thereby effectively preventing infectious diseases caused by the corresponding virus.

(c) Furthermore, the green tea extract of the present invention has no toxicity, thereby producing a safe virus vaccine, and unlike a chemical substance-based preparation procedure, a dialysis process is not needed, and thus, the preparation method has excellent economical efficiency.

### BRIEF DESCRIPTION OF THE DRAWINGS

FIG. 1a shows the results of SDS-PAGE analysis after nucleoprotein of A/Puerto Rico/8/34(H1N1) virus was reacted with a green tea extract.

FIGS. 1b, 1c, and 1d show the results of SDS-PAGE analysis after Lysyl-tRNA synthetase (LysRS)-HA fusion proteins of A/Korea/01/2009(H1N1) virus were reacted with a green tea extract.

FIG. 1e shows the results of SDS-PAGE analysis after hemagglutinin protein of A/Puerto Rico/8/34(H1N1) virus was reacted with EGCG.

FIG. 1f shows the results of LCMS/MS analysis of hemagglutinin protein reacted with EGCG.

FIG. 2a shows virus replication activity and hemagglutination activity when a mixture of equal amounts of virus (5×10⁷ PFU/ml) and a green tea extract (1 mg/ml) was incubated according to the temperature.

FIG. 2b shows virus replication activity and hemagglutination activity when various concentrations of virus (5×10⁷, 1×10⁸, and 5×10⁸ PFU/ml) was mixed with a green tea extract (1 mg/ml) in equal amounts and the mixture was incubated.

FIG. 2c shows virus replication activity and hemagglutination activity when virus (5×10⁷ PFU/ml) was mixed with various concentrations of a green tea extract (0.1, 0.5, 1 mg/ml) in equal amounts, and incubated. The dotted lines represent the detection limit. The detection limit for virus replication activity assay was 5 PFU/ml and the detection limit for hemagglutination activity assay was 2 HAU/ml.

FIG. 3a shows the results of plaque assay performed to investigate whether virus was completely inactivated.

FIG. 3b shows the results of confirming that GT-V lost its ability to infect chicken embryos.

FIG. 4a shows toxicity test results of GT-V. FIG. 4b shows toxicity test results of a green tea extract.

FIG. 5a shows hemagglutination inhibition (HI) test results of GT-V. FIG. 5b shows virus neutralization test (VNT) results of GT-V. The dotted lines represent the detection limit. The detection limit for HI assay was 8 (HI titer) and the detection limit for neutralization ability assay was 20 (NT titer).

FIG. 6 shows the results of analysis of protective effect of GT-V against virus challenge.

FIG. 7 shows the results of confirming the inhibition of infectious virus replication in the lung of mice immunized with GT-V. The dotted lines represent a detection limit of 50 PFU/ml.

FIG. 8 shows toxicity test results of dialyzed or non-dialyzed GT-V.

FIG. 9 shows the results of SDS-PAGE assay and Western blotting assay after infectious bronchitis virus (IBV) strain M41 was reacted with a green tea extract.

FIG. 10 shows the result of dot-immunoblot assay (DIB) to confirm that IBV was inactivated by GT.

FIG. 11 shows the results of analysis of antibody titer of IgG in the serum of mice immunized with GT-IBV.

FIG. 12a shows the results of dot-immunoblot assay (DIB) of neutralization antibody of mouse serum collected at 2 weeks after GT-IBV inoculation.

FIG. 12b shows the results of DIB detection of neutralizing antibody of mouse serum collected at the 6th week after GT-IBV inoculation.

FIG. 13 shows the results of SDS-PAGE analysis after hRBD-L1 fusion protein of human papillomavirus was reacted with a green tea extract.

FIG. 14 shows the result of SDS-PAGE analysis after hRBD-NoV VP1 fusion protein of norovirus was reacted with a green tea extract.

### SUMMARY

Hereinafter, the present invention will be described in detail with reference to examples. These examples are only for illustrating the present invention more specifically, and it will be apparent to those skilled in the art that the scope of the present invention is not limited by these examples.

### EXAMPLES

Materials

Cell Lines

Madin-Darby canine kidney (MDCK) and Vero cells were obtained from American Type Culture Collection (ATCC), and the cells were incubated using 10% fetal bovine serum (FBS, HyClone, US) and minimal essential medium (MEM, HyClone, US) under the conditions of 5% CO₂ and 37° C.

Virus and Green Tea Extract

A/Puerto Rico/8/34 (H1N1) virus was inoculated into 11-day-old specific pathogen free (SPF) chicken embryos, and incubated for 2 days in a 37° C. incubator. Then, an allantoic fluid was collected, followed by impurity removal therefrom, and stored in −80° C. refrigeration equipment.

Infectious bronchitis virus (IBV) strain M41 was inoculated into 11-day-old specific pathogen free (SPF) chicken embryos, and incubated for 2 days in a 37° C. incubator. Then, an allantoic fluid was collected, followed by impurity removal therefrom, and stored in −80° C. refrigeration equipment.

L1 protein (HPV 16L1), which is type 16 virus-like particle (VLP)-derived enveloped protein, was used for human papillomavirus (HPV).

VP1 (NoV VP1), which is a structural protein of Hu/GII.4/Hiroshima/55/2005/JPN strain, was used for norovirus (NoV).

For a green tea extract, powdered green tea (100% green tea, AmorePacific, Korea) was dissolved in tertiary distilled water, and then purified using a 0.2 μm syringe filter.

EGCG (EGCG 98%, Changsha Sunfull Bio-tech, China) was dissolved in tertiary distilled water, and then purified using a 0.2 μm syringe filter.

Methods and Results

Analysis of Influenza Protein Treated with Green Tea Extract

To investigate the effect of the green tea extract according to the present invention on an influenza protein, nucleoprotein (NP) of A/Puerto Rico/8/34 (H1N1) virus was reacted with the green tea extract, and then analyzed through SDS-PAGE.

First, a nucleic acid sequence encoding the nucleoprotein was inserted into pGE-LysRS(3) vector, expressed in E. coli, and then separated and purified using nickel chromatography. Next, 1 μg/10 μl purified nucleoprotein was reacted with 10, 100, and 1000 μg/10 μl green tea extract at room temperature for 6 hours. Thereafter, the nucleoprotein treated with the green tea extract was loaded on 10% PAGE gel to perform electrophoresis, and the gel was stained with Coomassie-blue to identify stained protein bands.

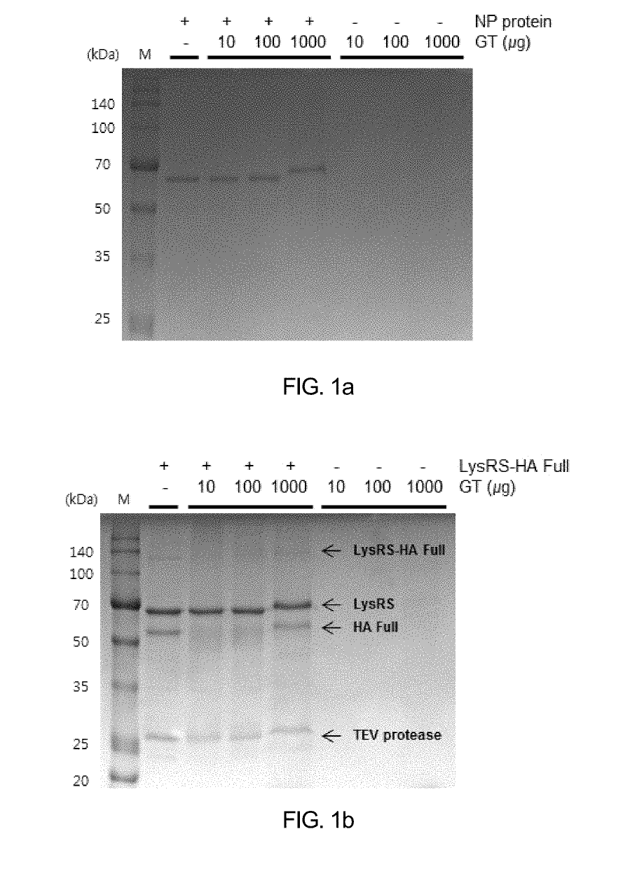

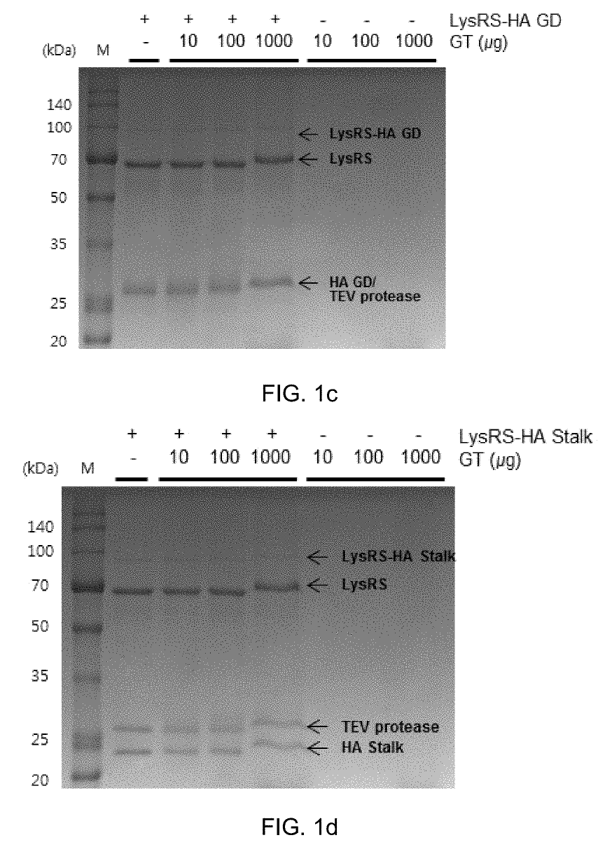

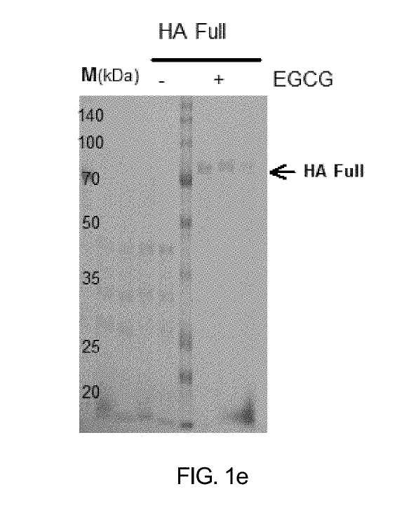

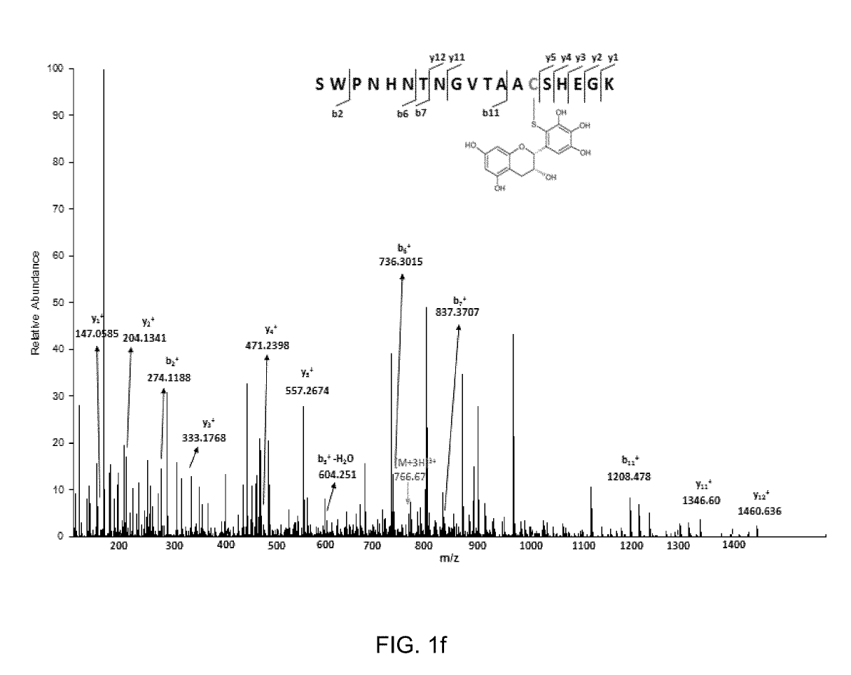

As a result, it was confirmed that, in a group treated with 1000 μg of green tea extract, the molecular weight of the nucleoprotein was increased due to the binding of the protein and the green tea extract, leading to an increase in band size, but there was no significant difference at low concentrations (FIG. 1a).

Then, Lysyl-tRNA synthetase (LysRS)-HA fusion protein of A/Korea/01/2009(H1N1) virus was reacted with a green tea extract, and then analyzed through SDS-PAGE.

A nucleic acid sequence encoding LysRS-HA fusion protein was inserted into pGE-LysRS(3) vector, expressed in E. coli, and then separated and purified using nickel chromatography. The LysRS-HA fusion protein may have three different structures, HA globular domain (LysRS-HA GD) and HA stalk region (LysRS-HA Stalk), which correspond to a head part of hemagglutinin, and HA full (LysRS-HA full) of HA globular domain plus HA stalk region. The respective LysRS-HA Full, LysRS-HA GD, and LysRS-HA Stalk fusion proteins were treated with TEV protease (Invitrogen, US) to digest LysRS proteins, and then 1 μg/10 μl LysRS-HA Full, LysRS-HA GD, and LysRS-HA Stalk were reacted with 10, 100, and 1000 μg/10 μl green tea extract at room temperature for 6 hours. Thereafter, the reaction products were loaded on 10% PAGE gel to perform electrophoresis, and the gel was stained with Coomassie-blue to identify stained protein bands.

As a result, it was confirmed that, in a group treated with green tea extract (1000 μg), the molecular weights of all of the LysRS-HA full protein, HA full protein, and HA globular and HA stalk were increased, leading to an increase in band size, but there was no significant difference at low concentrations. Therefore, it was confirmed that the green tea extract according to the present invention bound to virus full proteins (FIGS. 1b to 1d).

Assay of Influenza Protein Treated with EGCG

Hemagglutinin (HA) of A/Puerto Rico/8/34(H1N1) virus was reacted with EGCG, followed by analysis through SDS-PAGE

Hemagglutinin was expressed in human cells. 2 μg/10 μl hemagglutinin was reacted with 100 μg/10 μl EGCG at room temperature for 2 hours. Thereafter, hemagglutinin treated with EGCG was loaded on 10% PAGE gel to perform electrophoresis, and the gel was stained with Coomassie-blue to identify stained protein bands.

As a result, it was confirmed that the reaction with EGCG increased the molecular weight of the protein and thus the band size was increased. Therefore, it was confirmed that EGCG according to the present invention bound to the hemagglutinin protein of the influenza virus (FIG. 1e).

In addition, the hemagglutinin protein reacted with EGCG was analyzed through liquid chromatography mass spectrometry (LCMS/MS). The protein bands stained with Coomassie blue were separated by in-gel digestion, subjected to alkylation and de-staining processes, and then prepared into peptide fragments using trypsin. The prepared peptide fragments were analyzed using LCMS/MS [(Q-Exactive mass spectrometer (Thermo Fisher Scientific, Bremen, Germany) coupled with an Easy-nLC system (Thermo Fisher Scientific, Odense, Denmark)].

As a result, it was confirmed that EGCG is modified in the form of dihydro epigallocatechin (C₁₅H₁₁O₆) and bound to the cysteine residue, which is the 152nd amino acid of the influenza hemagglutinin protein (FIG. 1f).

Inactivation Effect of Green Tea Extract on Influenza Virus

In order to investigate the inactivation effect when influenza was directly treated with a green tea extract, virus replication activity, hemagglutination activity, and growth kinetic tests were carried out under various conditions.

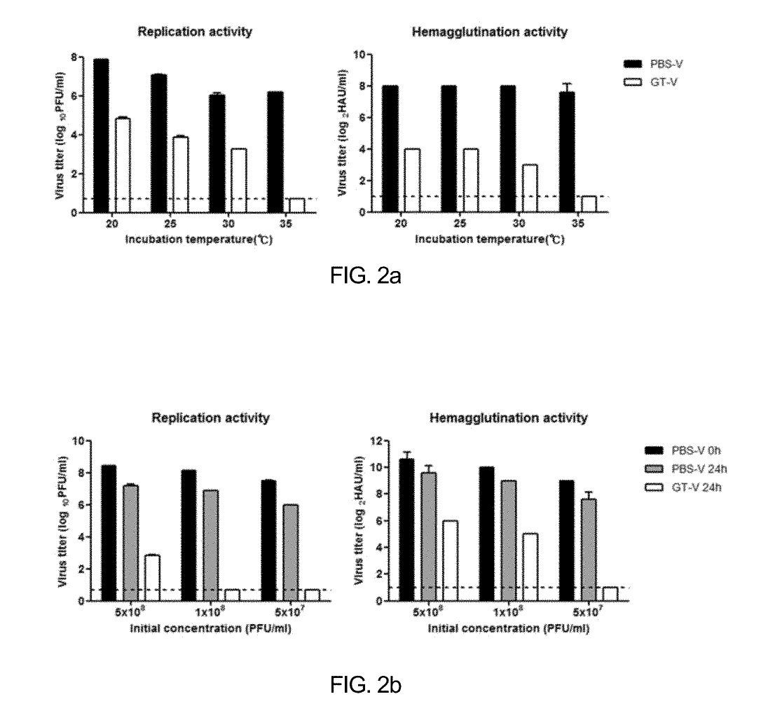

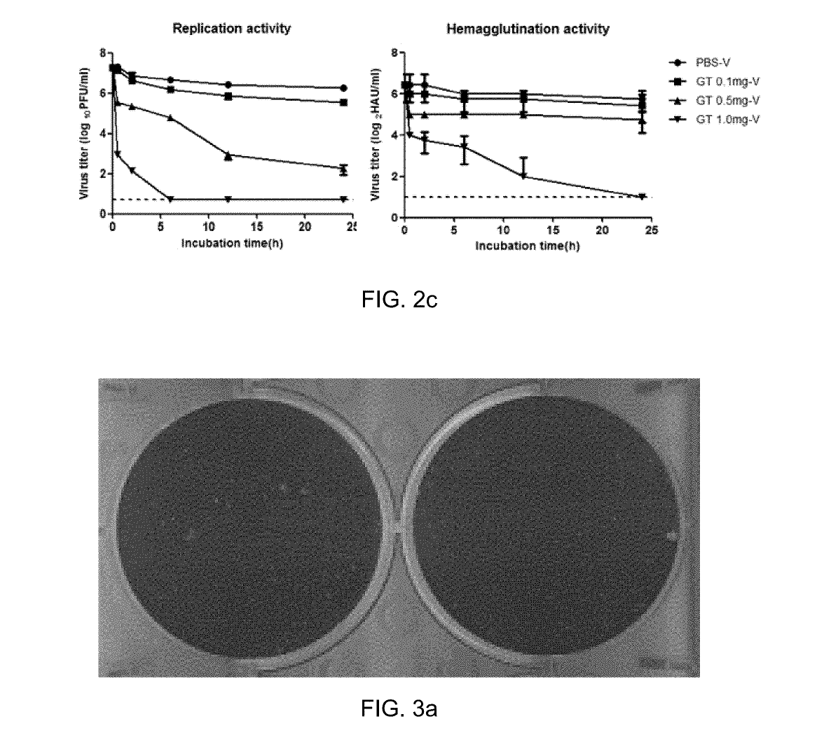

First, in order to investigate the degree of inactivation depending on the temperature, virus (5×10⁷ PFU/ml) was mixed with a green tea extract (1 mg/ml) in equal amounts, followed by incubation in a constant-temperature water bath at 20, 25, 30, and 35° C. The mixed solution was inoculated on a 12-well plate in which MDCK cells have been cultured, and the virus titer was examined by plaque assay. As a result, it was confirmed that the virus replication activity was decreased by about 3 log₁₀ PFU/ml with increasing temperature, and virus replication was all inhibited at 35° C. It was confirmed that the hemagglutination activity was also decreased depending on the temperature and the hemagglutination activity was all inhibited at 35° C. (FIG. 2a).

Then, in order to investigate the degree of inactivation according to the virus titer, virus with various titers (5×10⁷, 1×10⁸, and 5×10⁸ PFU/ml) and a green tea extract (1 mg/ml) were mixed in equal amounts, followed by incubation in a constant-temperature water bath at 35° C. at which the virus has been effectively inhibited in the previous test. The mixed solution was inoculated on a 12-well plate in which MDCK cells have been cultured, and the virus titer was examined by plaque assay. As a result, it was confirmed that the virus replication activity was increased as the titer of virus was higher, and the virus replication activity was inhibited at both of the titers of 1×10⁸ PFU/ml and 5×10⁷ PFU/ml. It was confirmed that hemagglutination activity was also decreased depending on the titers and hemagglutination activity was all inhibited at the titers of 5×10⁷ PFU/ml (FIG. 2b).

On the basis of the above test results, 5×10⁷ PFU/ml virus and the green tea extract with various concentrations (0.1, 0.5, and 1 mg/ml) were mixed in equal amounts, and then the growth kinetic of virus depending on the time was examined while the mixture was incubated at 35° C. for 24 hours. The mixed solution was inoculated on a 12-well plate in which MDCK cells were cultured, and the virus titer was examined by plaque assay. As a result, it was confirmed that virus replication activity was decreased according to the concentration and time, and for 1 mg/ml green tea extract, virus replication activity was all inhibited within 6 hours. The hemagglutination activity was also decreased as the concentration of the green tea extract was increased, and the treatment time was longer, and for 1 mg/ml green tea extract, the hemagglutination activity was all inhibited 24 hours after the treatment (FIG. 2c).

Preparation of Inactivated Influenza Virus Vaccine (GT-V)

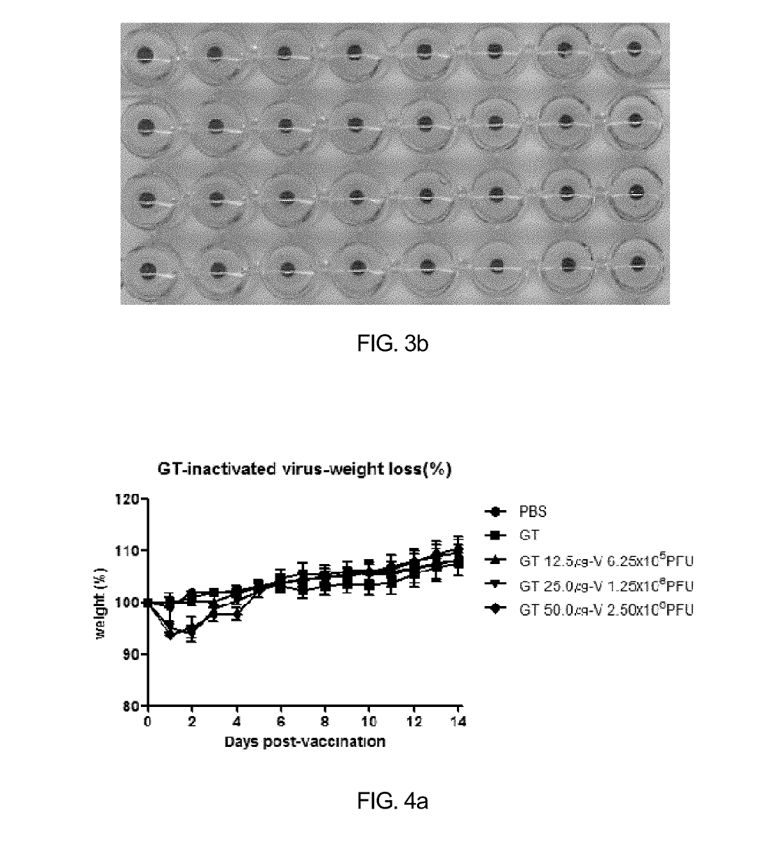

On the basis of the above test results, 5×10⁷ PFU/ml A/Puerto Rico/8/34(H1N1) and 1 mg/ml green tea extract were mixed in equal amounts, followed by incubation at 35° C. for 24 hours, thereby preparing an influenza inactivated vaccine (GT-V) after the treatment with the green tea extract. In order to investigate whether the virus was completely inactivated, the vaccine was inoculated into MDCK cells, followed by plaque assay. As a result, it was confirmed that no plaque was generated, indicating that viral activity was abolished (FIG. 3a). For more accurate validation, the prepared GT-V stock solution was inoculated into 11-day-old embryos and cultured at 37° C. for 2 days, and then an allantoic fluid was collected to examine hemagglutination ability. As a test result, hemagglutination ability was not observed, confirming that the influenza inactivated vaccine (GT-V) of the present invention lost its ability to infect chicken embryos (FIG. 3b).

Investigation of Toxicity of Inactivated Influenza Virus Vaccine

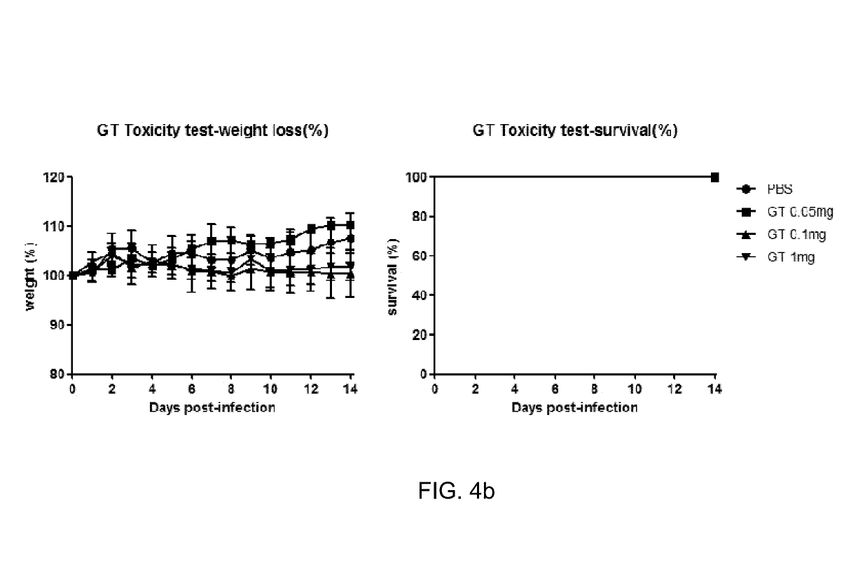

In order to investigate toxicity of the GT-V prepared above, mice were intraperitoneally administered with GT-V (200 μl/mice) with various concentrations (GT (Green tea) 12.5 μg-V (virus) 6.25×10⁵ PFU, GT 25.0 μg-V 1.25×10⁶ PFU, and GT 50.0 μg-V 2.50×10⁶ PFU) and PBS together with alum (100 μl) as an adjuvant, and the body weight change was monitored for 14 days. Although a slight weight loss was observed until 2 days after the inoculation, the weight loss was about 5% compared with a control group, indicating no significant difference, and then the body weight was continuously recovered, and returned to the normal weight after day 5. Therefore, it was confirmed that GT-V of the present invention showed no toxicity in animal test results (FIG. 4a).

In order to examine toxicity of only the green tea extract, four mice per group were intraperitoneally injected (100 μl) with a green tea extract (0.05, 0.1, 1 mg) and PBS. The mice were observed for the weight loss change and survival rate for 14 days. As a result, compared with a mouse group (control group) administered with PBS, all mouse groups administered with green tea extract showed no significant body weight loss at all doses, and showed 100% survival rates. In the GT-V animal test, the highest dose of the green tea extract was 0.05 mg, and it was confirmed that toxicity was not observed even when mice were administered with a green tea extract of 1 mg, which is 20-fold higher than 0.05 mg (FIG. 4b).

Investigation of Immunogenicity of GT-V

GT-V Inoculation and Blood Collection

In order to investigate immunogenicity and protective effect of GT-V, five mice per group were intraperitoneally administered with 100 μl of GT-V (100 μl/mice) with various concentrations (GT 12.5 μg-V 6.25×10⁵ PFU, GT 25.0 μg-V 1.25×10⁶ PFU, GT 50.0 μg-V 2.50×10⁶ PFU) together with 100 μl of alum as an adjuvant, and additionally inoculated at the same concentrations after 2 weeks. The mouse body weight change was observed daily for 2 weeks after the inoculation, and after 2, 4, and 6 weeks of the first inoculation, blood was collected, and subjected to centrifugation to collect only serum, which was then used for immunogenicity analysis.

All the test procedures were carried out according to the guidelines of the Institutional Animal Care and Use Committee (IACUC) of Yonsei Laboratory Animal Research Center.

Hemagglutination Inhibition Assay

In order to analyze hemagglutination inhibition characteristics of GT-V, hemagglutination inhibition (HI) analysis was performed. First, the serum was treated with a receptor destroying enzyme, which was then inactivated by heating at 56° C. for 1 hour. Then, 25 μl of the serum was diluted 2-fold serially with PBS in a 96-well plate. Then 4 HAU/25 μl of the same wild type of A/Puerto Rico/8/34 (H1N1) virus was added to the diluted serum, followed by incubation at 37° C. for 1 hour. Thereafter, 50 μl of 1% chicken red blood cells (cRBC, chicken RBC) was added, followed by incubation at 4° C. for 1 hour, and then the highest dilution rate for inhibiting hemagglutination activity was calculated.

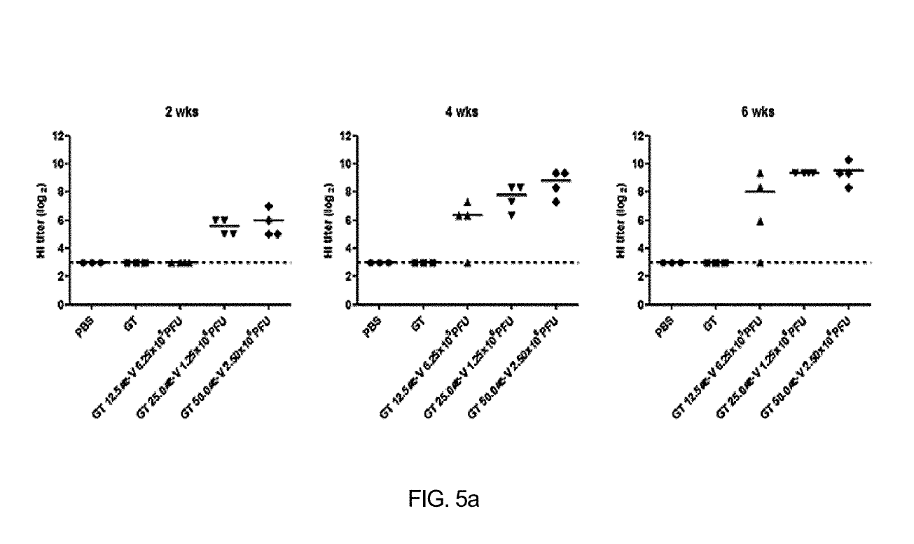

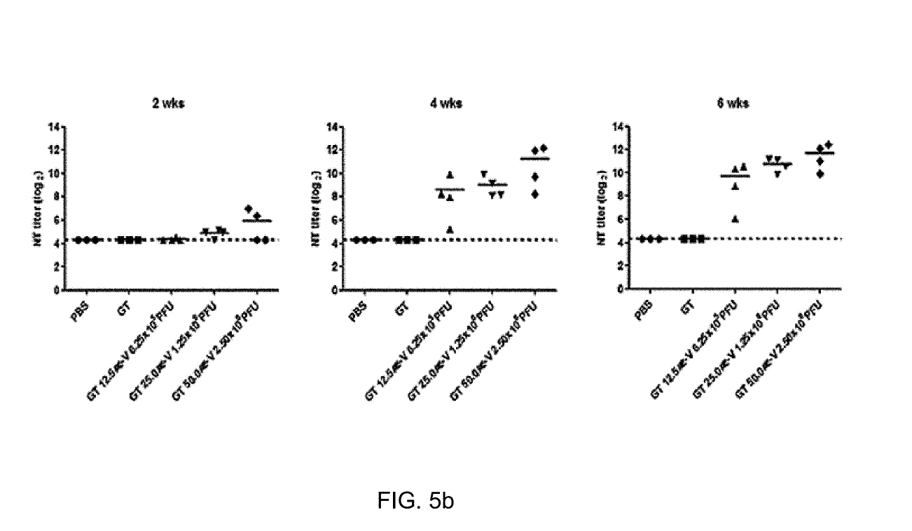

As a result, it was confirmed that the HI titer was not shown at the lowest inoculation concentration at the 2nd week, but after the additional inoculation, the HI titer was significantly increased, and was highly induced by the concentration at each week. It was confirmed that the HI titer showed the highest value at the 6th week, confirming that the immune-induced response by GT-V of the present invention was maintained for 6 weeks or longer (FIG. 5a).

Virus Neutralization Assay

In order to investigate virus neutralization ability of the serum of mice inoculated with GT-V, virus neutralization test (VNT) was carried out. First, the serum of a mouse inoculated with GT-V, the serum being collected in the above example, was inactivated by heating at 56° C. for 1 hour. Then, 25 μl of each serum was diluted 2-fold serially with PBS in a 96-well plate. Next, 100 PFU/100 μl of virus was added to the diluted serum, followed by a neutralization reaction at 37° C. for 1 hour. Thereafter, the virus and serum, which had been subjected to the neutralization reaction, were inoculated on a 12-well plate in which MDCK cells were cultured, and then plaque assay was performed. The dilution ratio showing a 50% plaque reduction compared with a control group was calculated.

As a result, it was confirmed that the neutralization titer (NT titer) was hardly increased at the 2nd week after the first inoculation, but the neutralization titer was greatly increased after the additional inoculation, and further increased at the 6th week to maintain the immune response (FIG. 5b).

Analysis of Protective Effect of GT-V Against Virus Challenge

Mice inoculated with GT-V (GT 12.5 μg-V 6.25×10⁵ PFU, GT 25.0 μg-V 1.25×10⁶ PFU, and GT 50.0 μg-V 2.50×10⁶ PFU) were additionally inoculated in equal amounts after two weeks. At the 4th week after the additional inoculation, A/Puerto Rico/8/34 (H1N1) virus was intranasally challenged in 10⁴ PFU/50 μl, which was a concentration of 10 times the 50% mortality rate (10 MLD₅₀), and then the body weight change and survival rate were monitored for 2 weeks after the challenge.

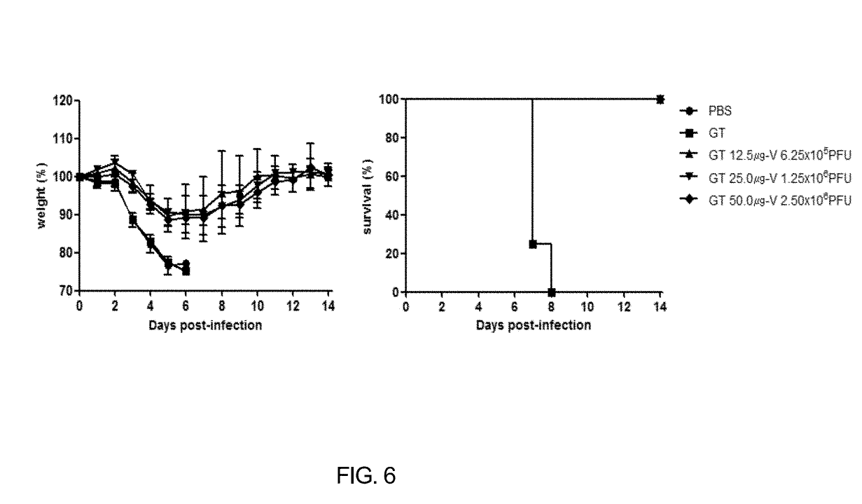

As a result, the mice inoculated with GT-V showed a body weight loss of about 10% until the 6th day after the challenge, but thereafter, the body weight was recovered. A control group not inoculated with GT-V showed a rapid body weight loss, and then all mice were dead on the 6th day after the challenge. Regardless of the inoculation concentration of GT-V, survival rate was 100% even in the group inoculated with the lowest concentration of GT-V (FIG. 6).

Investigation of Inhibition of Virus Replication in Lung

In order to further investigate protective effect of GT-V against fatal influenza virus infection, mice were inoculated twice, and 4 weeks later, intranasally challenged with 10 MLD₅₀ (10⁴ PFU/50 μl) of A/Puerto Rico/8/34 (H1N1) virus as in the above example, and 2, 4 and 6 days later, the mice were sacrificed to collect lungs thereof. The collected lungs were put into 500 ml of PBS, followed by disruption, and then centrifuged to separate only supernatant. The separated supernatant was inoculated into MDCK cells, and plaque analysis was performed to check the titer of virus present in the lungs of mice.

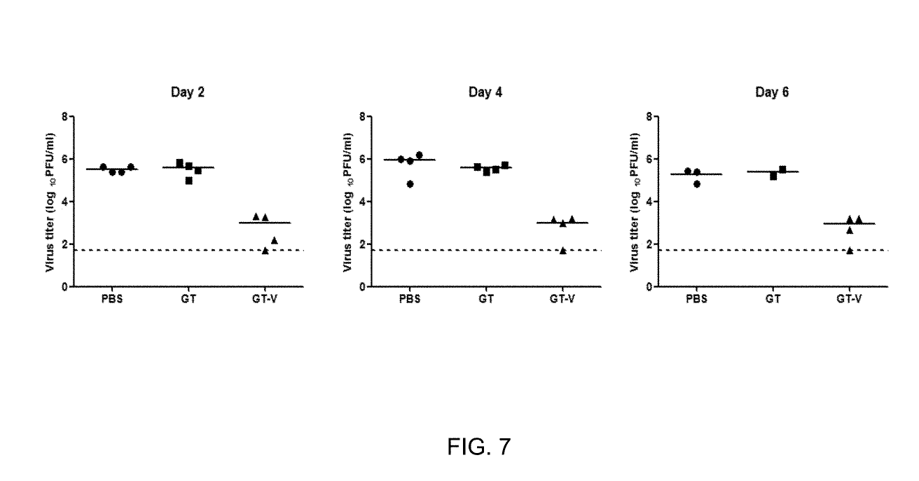

As a result, the infectious virus identified in the lungs of mice inoculated with GT-V showed a virus titer, which was approximately 10³ times lower than that in the mice not inoculated with GT-V. This value was observed on even day 2 and day 4 as well as day 6 after the inoculation, and the viral replication was not completely inhibited, but a sufficiently low inhibition value was confirmed (FIG. 7).

Investigation of Need of Dialysis

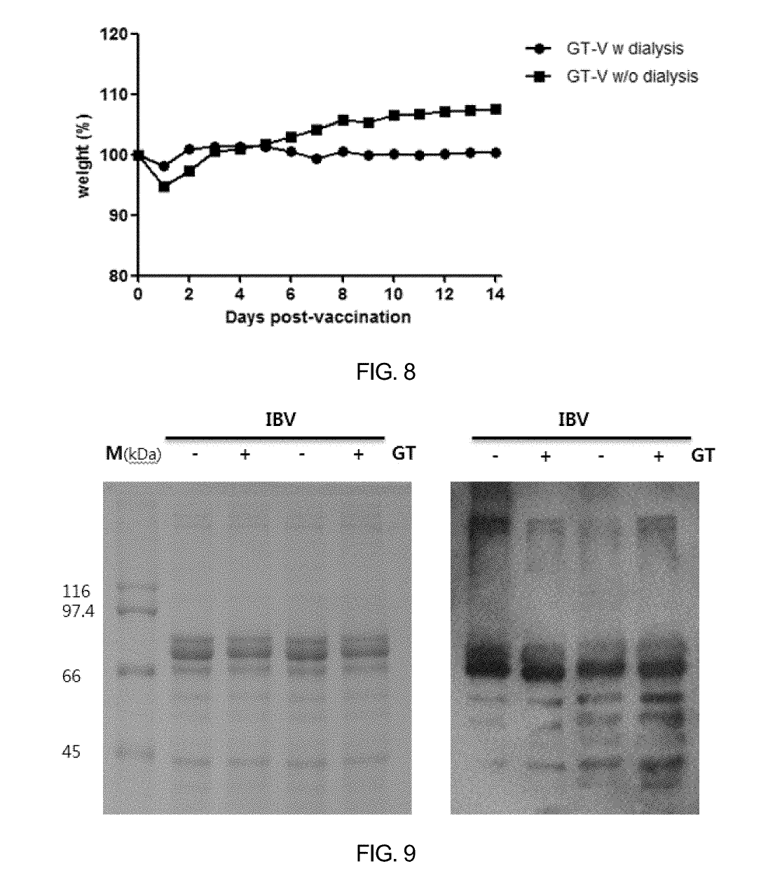

In order to investigate whether dialysis was needed when virus was inactivated by the green tea extract according to the present invention like in a case where the virus was inactivated by formaldehyde, the toxicity of GT-V subjected to a dialysis procedure for removing the green tea extract and GT-V not subjected to a dialysis procedure was tested by the same method as in the foregoing GT-V toxicity test, and the results were compared. The mixed solution of the green tea extract and the virus was dialyzed with PBS buffer (pH 7.4) at 4° C. for 24 hours. As a result, there was no significant difference in body weight between a mouse group inoculated with GT-V subjected to a dialysis procedure and a mouse group inoculated with GT-V not subjected to a dialysis procedure (FIG. 8).

Therefore, GT-V of the present invention does not require a dialysis process, indicating that GT-V of the present invention is highly economical in manufacturing vaccines.

Analysis of Coronavirus Treated with Green Tea Extract

In order to investigate the effect of the green tea extract according to the present invention on coronavirus, infectious bronchitis virus (IBV) strain M41 was reacted with the green tea extract, followed by analysis through SDS-PAGE. 100 μl of virus (10⁶⁵ EID₅₀/ml) was reacted with 100 μl of the green tea extract (10 mg/ml) at room temperature for 2 hours. Thereafter, the reaction product was loaded on 8% PAGE gel, followed by electrophoresis. Thereafter, the gel was stained with Coomassie-blue to identify stained protein bands, and, at the same time, western blotting was performed. The protein bands on the gel were transferred to polyvinylidene fluoride (PVDF) membrane, and for the reduction of non-specific reactions, the membrane was blocked with 5% skim milk, and then washed with TBST. The serum of mice inoculated with IBV was diluted to 1:1000, and used as primary antibody with respect to the membrane. The membrane was washed with TBST, and horseradish peroxidase (HRP)-conjugated anti-mouse IgG (HRP-conjugated anti-mouse IgG) was diluted to 1:10000, and thus, the membrane was treated with secondary antibody. The membrane was washed with TBST, and then treated with WEST-ZOL plus Western Blot Detection System (iNtRON, Korea), and developed on X-ray film.

As a result, it was confirmed that the reaction with the green tea extract increased the molecular weight of the protein, and thus the band size was increased. Therefore, it was confirmed that the green tea extract according to the present invention bound to the coronavirus protein (FIG. 9).

Preparation of Inactivated Coronavirus Vaccine (GT-IBV)

Infectious bronchitis virus (IBV) strain M41 (10⁶⁵ EID₅₀/ml) and a green tea extract (1 mg/ml) were mixed in equal amounts, followed by incubation at 35° C. for 24 hours, thereby preparing a green tea extract-treated corona inactivated vaccine (GT-IBV). In order to investigate whether the virus was completely inactivated, the GT-IBV stock solution was inoculated onto 11-day-old chicken embryos, followed by incubation at 37° C. for 2 days. Then, an allantoic fluid was collected, and dot-immunoblot assay (DIB) was performed for measuring residual amount of virus. The mixture of the virus and green tea extract was dispensed in 200 μl for each nitrocellulose paper (NC paper), followed by vacuum treatment for 10-15 minutes and then washing. The nitrocellulose paper was blocked with 3% bovine serum albumin (BSA) at 37° C. for 2 hours, and then, the serum of mice inoculated with IBV was diluted to 1:1000, followed by incubation at 37° C. for 30 minutes. The reaction product was washed three times with TBST and treated with biotinylated anti-mouse IgG, followed by incubation at 37° C. for 30 minutes. The reaction product was washed three times with TBST and treated with biotin and avidin-conjugated peroxidase complex (ABC) kit, followed by incubation at 37° C. for 30 minutes. The reaction product was washed three times with TBST, treated with diaminobenzidine to perform color development for 1 minute, and washed with flowing water, followed by drying, to investigate staining or non-staining.

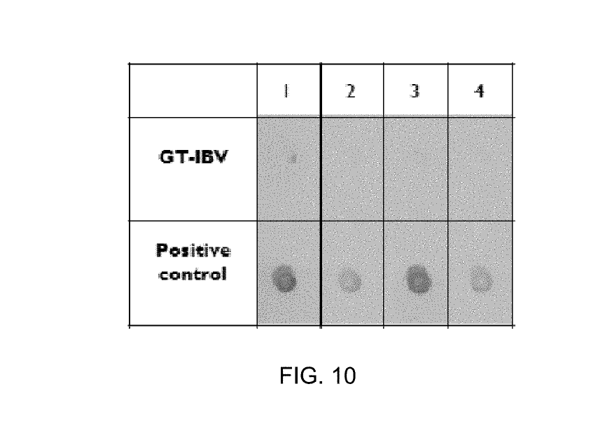

As a result, an allantoic solution of chicken embryos inoculated with GT-IBV of the present invention was not stained with IBV antibody, confirming that IBV activity was lost (FIG. 10).

Investigation of Immunogenicity of GT-IBV

GT-IBV Inoculation and Blood Collection

In order to investigate the immunogenicity of GT-IBV of the present invention, four mice per group were intraperitoneally administered with 100 μl of GT-V with various concentrations (GT 12.5 μg-IBV 1.25×10^{4.5} EID₅₀, GT 25.0 μg-IBV 2.50×10^{4.5} EID₅₀, and GT 50.0 μg-V 5.0×10^{4.5} EID₅₀) and 100 μl of alum as an adjuvant. After 2 weeks, additional inoculation was carried out at the same concentrations. Blood was collected at 2 weeks and 6 weeks after the first inoculation, and centrifuged to collect only serum, which was then used for immunogenicity analysis.

All the test procedures were carried out according to the guidelines of the Institutional Animal Care and Use Committee (IACUC) of Yonsei Laboratory Animal Research Center.

Analysis of IgG Titer

The serum of mice inoculated with GT-V of the present invention was subjected to ELISA analysis. Wild-type (WT) IBV virus (10⁶⁵ EID₅₀/ml) was dispensed into a 96 well plate at 100 μl per each well, followed by coating at 4° C. for one day. The virus-coated plate was washed three times with Tris-HCl (pH 7.4) and blocked with 1 BSA at room temperature for 1 hour. After washing in the same manner, the serum of mice inoculated with GT-V of the present invention was initially diluted to 1:200, then 2-fold serially diluted, and dispensed at 100 μl/well in a 96-well plate and treated at room temperature for 1 hour. The reaction product was washed by the same method, and then treated with 1:1000-diluted HRP-conjugated anti-mouse IgG (Mab) at 100 μl/well at room temperature for 1 hour. The reaction product was washed by the same method, and then treated with TMB solution at 100 μl/well at room temperature for 30 minutes. The reaction was stopped by treatment with 2N H₂SO₄, and analyzed by using a spectrometer at 450 nm.

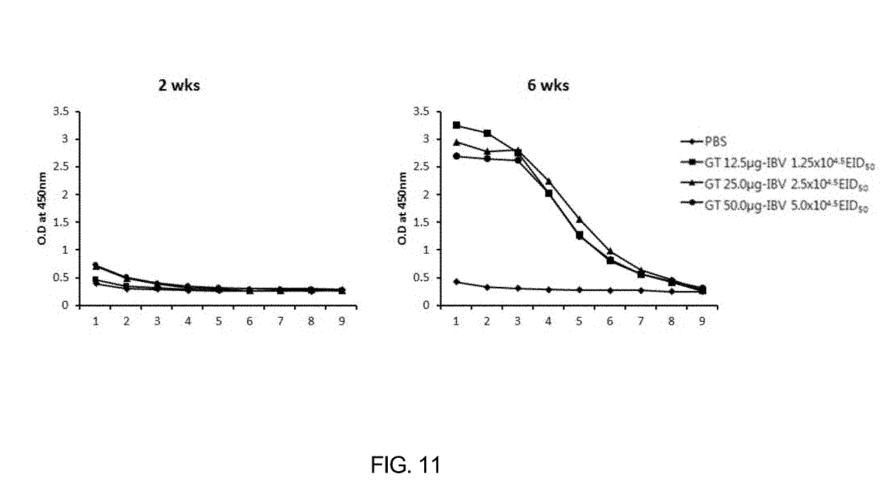

As a result, it was confirmed that IgG antibody was hardly produced at the 2nd week after the first inoculation, but the antibody titer was significantly increased at the 6th week after the second inoculation, and thus, the antibody was sufficiently produced at each GT-IBV inoculation concentration (FIG. 11).

Virus Neutralization Assay

In order to investigate virus neutralization ability of the serum of mice inoculated with GT-IBV, virus neutralization test (VNT) was carried out. First, 100 μl of 10² EID₅₀/ml IBV strain was reacted with 100 μl of serum (10⁻³, 10⁻⁴, 10⁻⁵ dilution) at 37° C., the serum being collected, on the 2nd week and the 6th week, from mice inoculated with GT 12.5 μg-IBV 1.25×10^{4.5} EID₅₀, GT 25.0 μg-IBV 2.50×10^{4.5} EID₅₀, and GT 50.0 μg-V 5.0×10^{4.5} EID₅₀. Then, three chicken embryos were inoculated with each of the mixtures, followed by incubation at 37° C. for 3 days. Thereafter, an allantoic solution of each chicken embryo was collected, and dot-immunoblot assay (DIB) was performed by the same method as in the neutralization assay.

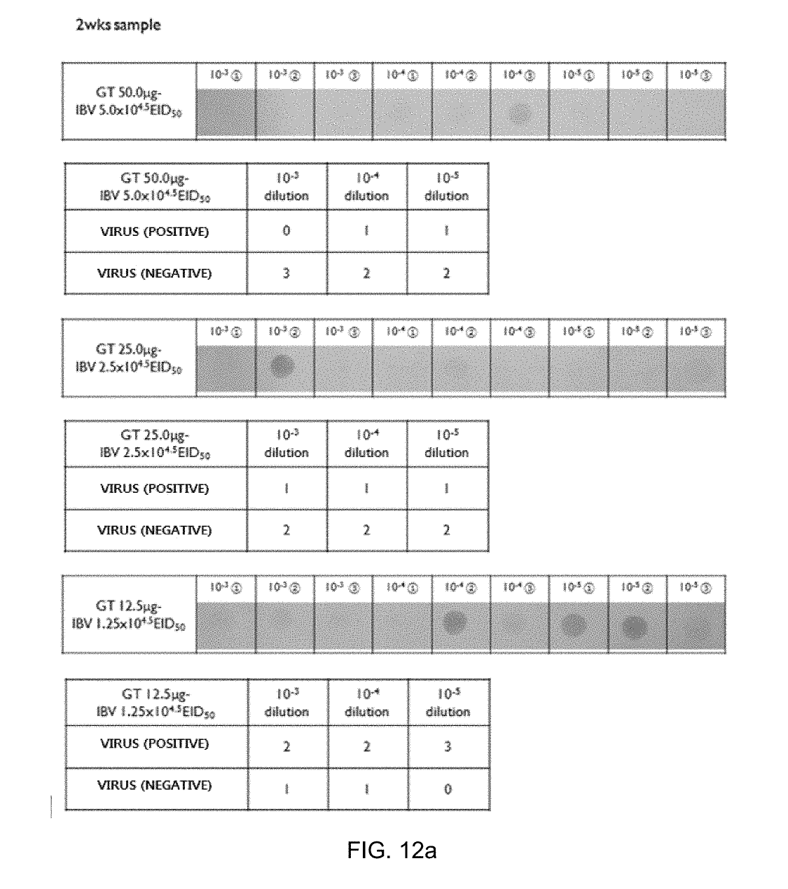

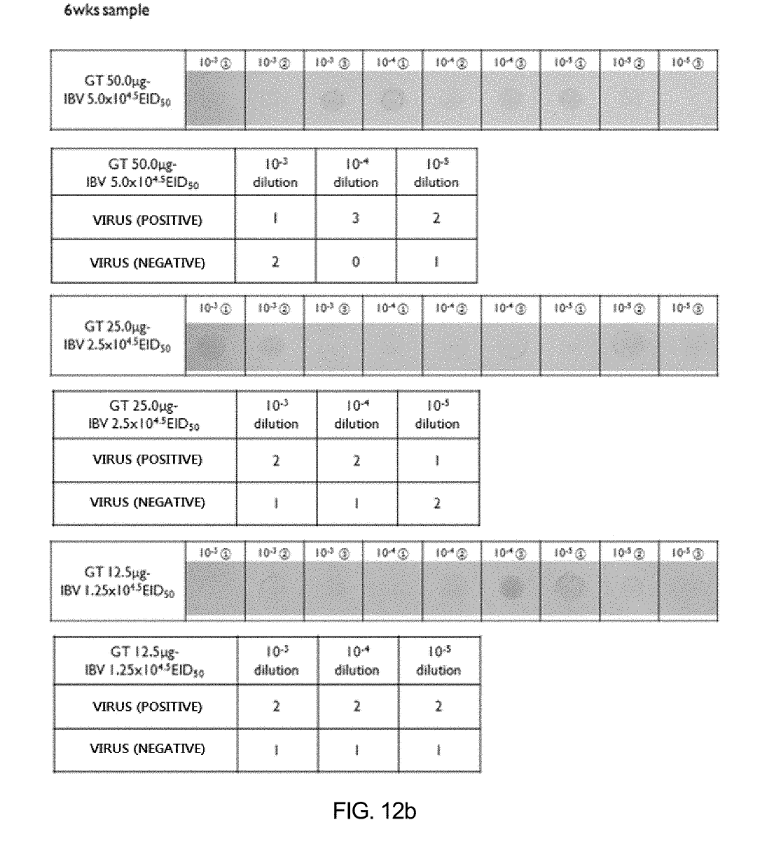

As a result, it was confirmed that the negative viruses accounted for 78% in the GT 12.5 μg-IBV 1.25×10^{4.5} EID₅₀ group, 67% in the GT 25.0 μg-IBV 2.50×10^{4.5} EID₅₀ group, and 22% in the GT 50.0 μg-V 5.0×10^{4.5} EID₅₀ group (FIG. 12a). In addition, it was confirmed that negative viruses accounted for 33% in the GT 12.5 μg-IBV 1.25×10^{4.5} EID₅₀ group, 44% in the GT 25.0 μg-IBV 2.50×10^{4.5} EID₅₀ group, and 33% in the GT 50.0 μg-V 5.0×10^{4.5} EID₅₀ group (FIG. 12b). Therefore, it was confirmed that the serum of mice inoculated with coronavirus treated with a green tea extract could neutralize coronavirus.

Analysis of Non-Influenza Protein Treated with Green Tea Extract

Analysis of HPV Protein Treated with Green Tea Extract

In order to investigate the effect of the green tea extract according to the present invention on a non-influenza virus, LI protein (HPV 16L1), which is type 16 enveloped protein of human papillomavirus (HPV), was inserted into hRBD vector, expressed in E. coli, purified using nickel affinity chromatography, reacted with a green tea extract, and analyzed through SDS-PAGE. The hRBD-L1 fusion protein of human papillomavirus was treated with TEV protease (Invitrogen, USA) to digest hRBD. The L1 protein (2 μg/10 μl) was reacted with a green tea extract (10, 100, and 1000 μg/10 μl) of the present invention at room temperature for 2 hours. Thereafter, the reaction product was loaded on 10% PAGE gel to perform electrophoresis, and the gel was stained with Coomassie-blue to identify stained protein bands.

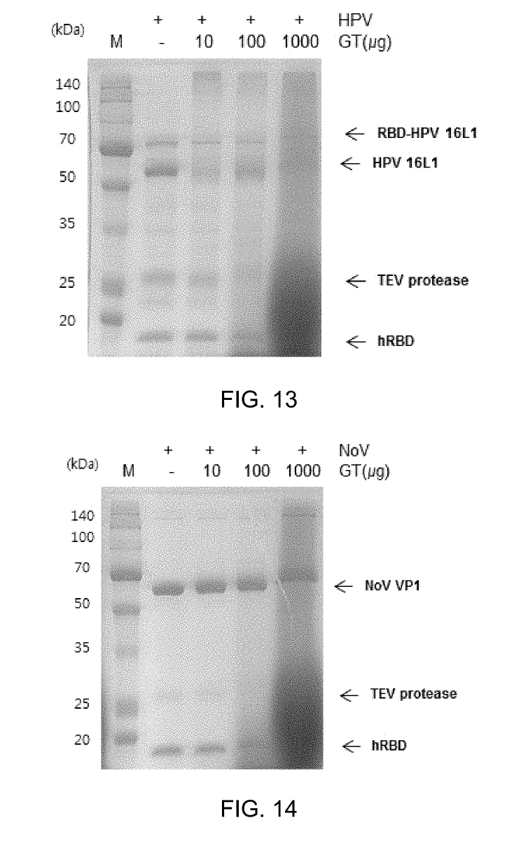

As a result, it was confirmed that there was no great difference at low concentrations, but when the L1 protein was reacted with 1000 μg/10 μl green tea extract, the molecular weight of the L1 protein was increased due to the binding between the protein and the green tea extract, and thus, the band size was increased. Therefore, it was confirmed that the green tea extract according to the present invention bound to the protein of human papillomavirus, which is a non-influenza virus (FIG. 13).

Analysis of Norovirus Protein Treated with Green Tea Extract

In order to investigate the effect of the green tea extract according to the present invention on another non-influenza virus, VP1 (NoV VP1), which is a structure protein of norovirus (NoV, Hu/GII.4/Hiroshima/55/2005/JPN), was inserted into hRBD vector, expressed in E. coli, purified using nickel affinity chromatography, reacted with a green tea extract, and analyzed through SDS-PAGE. The hRBD-Nov VP1 fusion protein of norovirus was treated with TEV protease (Invitrogen, USA) to digest hRBD. The VP1 protein (2 μg/10 μl) was reacted with the green tea extract (10, 100, and 1000 μg/10 μl) of the present invention at room temperature for 2 hours. Thereafter, the reaction product was loaded on 10% PAGE gel to perform electrophoresis, and the gel was stained with Coomassie-blue to identify stained protein bands.

As a result, it was confirmed that there was no great difference at low concentrations, but when the L1 protein was reacted with 1000 μg/10 μl green tea extract, the molecular weight of the VP1 protein was increased due to the binding between the protein and the green tea extract, and thus, the band size was increased. Therefore, it was confirmed that the green tea extract according to the present invention bound to the protein of norovirus, which is a non-influenza virus (FIG. 14).

Although the present invention has been described in detail with reference to specific features thereof, it will be apparent to those skilled in the art that this description is only for a preferred embodiment and does not limit the scope of the present invention. Thus, the substantial scope of the present invention will be defined by the appended claims and equivalents thereof.

1.-9. (canceled)

10. A method for preparing an inactivated virus vaccine, the method comprising:
(a) adding a green tea extract to a replicative virus, followed by mixing; and
(b) incubating a mixture of the virus and the green tea extract.

11. The method of claim 10, further comprising (c) adding an excipient.

12. The method of claim 11, further comprising (d) performing filtration, sterilization, and dilution.

13. The method of claim 10, wherein in step (a), the virus is an influenza virus and the virus and the green tea extract are mixed at a ratio of 5×1010 to 5×103 PFU:0.1-100 mg.

14. The method of claim 10, wherein in step (a), the virus is coronavirus and the virus and the green tea extract are mixed at a ratio of 1010 to 103 EID50:0.1-100 mg.

15. The method of claim 10, wherein the incubation in step (b) is carried out at a temperature of 15-50° C.

16. The method of claim 10, wherein the incubation in step (b) is carried out for 1 hour or longer.

17. A method for preventing a viral infectious disease, the method comprising administering, to a subject, the vaccine composition comprising a virus inactivated by a green tea extract.

18. The method of claim 18, wherein the viral infectious disease is caused by an infection with an influenza virus, coronavirus, human papillomavirus, or norovirus.

19. The method of claim 17, wherein the green tea extract comprises (−)-epigallocatechin gallate (EGCG).

20. The method of claim 17, wherein the virus is influenza A, B, or C virus.

21. The method of claim 20, wherein the influenza A virus is influenza A/H1N1, A/H3N2, A/H5N2, or A/H9N2 virus.

22. The method of claim 17, wherein the virus is coronavirus, human papillomavirus, or norovirus.

23. The method of claim 22, wherein the coronavirus is infectious bronchitis virus strain M41.

24. The method of claim 17, wherein the green tea extract binds to a protein of the virus.

25. The method of claim 24, wherein the virus is an influenza virus and the green tea extract binds to a nucleoprotein or hemagglutinin of the influenza virus.

26. The method of claim 25, wherein the hemagglutinin is a globular domain or a stalk region of the hemagglutinin.
